(19) United States
(12) Patent Application Publication
(10) Pub. No.: US 2013/0280318 A1
(43) Pub. Date: Oct. 24, 2013

(54) Tissue Scaffolds for Controlled Release of Active Agents

(71) Applicants: Helen H. Lu, New York, NY (US); Cevat Erisken, New York, NY (US)

(72) Inventors: Helen H. Lu, New York, NY (US); Cevat Erisken, New York, NY (US)

(73) Assignee: The Trustees of Columbia University in the City of New York, New York, NY (US)

(21) Appl. No.: 13/809,490

(22) PCT Filed: Jul. 12, 2011
(86) PCT No.: PCT/US11/43687

§ 371 Date: Jul. 15, 2013

Related U.S. Application Data
(60) Provisional application No. 61/399,519, filed on Jul. 12, 2010.

(51) Int. Cl. A61L 27/44

(52) U.S. Cl. CPC A61L 27/44
USPC 424/443; 514/8.9; 514/15.2; 424/93.7

(57) ABSTRACT

Tissue scaffolds of polymer microfiber or nanofiber mesh which release an active agent supportive of alignment, proliferation and matrix deposition of selected musculoskeletal cell over time are provided. Methods for production and use of these tissue scaffolds in treatment of musculoskeletal tissue injuries are also provided.

This patent application claims the benefit of priority from U.S. Provisional Application Ser. No. 61/399,519, filed Jul. 12, 2010, the entirety of the disclosure of which is explicitly incorporated by reference herein.

### STATEMENT REGARDING FEDERALLY SPONSORED RESEARCH OR DEVELOPMENT

This invention was made with government support under Grant Numbers AR 055280-02 (PECASE), AR 052402 and AR 056459-02 awarded by the National Institutes of Health—National Institute of Arthritis and Musculoskeletal and Skin Diseases (NIH-NIAMS). The government has certain rights in the invention.

### BACKGROUND

Injuries to soft connective tissues such as tendons or ligaments as well as cartilage are a common clinical problem.

Rotator cuff tears to the shoulder are among the most common soft connective tissue injuries, with greater than 75,000 repair procedures performed annually in the United States alone (Vitale et al. Elbow Surg. 2007 16:181). Clinical intervention is required because injuries to the rotator cuff do not heal, largely due to the complex anatomy and the extended range of motion of the shoulder joint, as well as the relative weakening and hypovascularization of the cuff tendons (Yamanaka, K. and Matsumoto, T. Chn. Orthop. Relat. Res. 1994 304:68-73; Dejardin et al. Am. J. Sports Med. 2001 29:175-184). Moreover, chronic degeneration increases both the frequency and size of cuff tears with age (Tempelhof et al. J. Shoulder Elbow Surg. 1999 8:296) and is considered the main contributing factor in the pathogenesis of rotator cuff tendon tears (Dejardin et al. Am. J. Sports Med. 2001 29:175-184; Soslowsky et al. J. Shoulder Elbow Surg. 2000 9:79). Early primary anatomic repair followed by carefully controlled rehabilitation is currently the treatment of choice for symptomatic rotator cuff tears (Dejardin et al. Am. J. Sports Med. 2001 29:175-184).

### SUMMARY

This application provides biomimetic tissue scaffolds for musculoskeletal tissue injuries designed to promote regeneration at tendon-bone interfaces through controlled release of active agents.

Accordingly, an aspect of this application relates to tissue scaffolds comprising a first phase of polymer microfiber and/or nanofiber mesh and an active agent released over time from the first phase which supports alignment, proliferation and matrix deposition of a selected musculoskeletal cell.

In one embodiment, the tissue scaffold may further comprise one or more additional phases of polymer microfiber and/or nanofiber mesh and an active agent released over time from the one or more additional phases which supports alignment, proliferation and matrix deposition of a selected musculoskeletal cell. In this embodiment, the different phases may contain the same active agent, different active agents or the same active agent in different concentrations. In this embodiment, the different phases may be seeded with the same selected musculoskeletal cell or stem cells which differentiate into the selected musculoskeletal cell, or different selected musculoskeletal cells.

In one embodiment the first phase and/or one or more additional phases of the tissue scaffold may further comprise an albumin.

Another aspect of the application relates to methods for producing tissue scaffolds which promote musculoskeletal cell proliferation, alignment and/or matrix production. In these methods, a selected musculoskeletal cell is seeded onto a tissue scaffold comprising a first phase of polymer microfiber and/or nanofiber mesh and an active agent released over time from the first phase which supports alignment, proliferation and matrix deposition of the selected musculoskeletal cells.

In one embodiment, the tissue scaffold comprises one or more additional phases of polymer microfiber and/or nanofiber mesh and an active agent released over time from the one or more additional phases which supports alignment, proliferation and matrix deposition of a selected musculoskeletal cell. In this embodiment, the different phases may contain the same active agent, different active agents or the same active agent in different concentrations.

In this embodiment, the different phases may be seeded with the same selected musculoskeletal cell or stem cells which differentiate into the selected musculoskeletal cell, or different selected musculoskeletal cells.

In one embodiment the first phase or one or more additional phases of the tissue scaffold produced may further comprise a purified albumin.

Another aspect of this application relates to tissue scaffolds for treatment of a musculoskeletal tissue injury produced in accordance with these methods.

Yet another aspect of this application relates to methods for treating musculoskeletal tissue injuries by surgically implanting at the site of musculoskeletal injury a tissue scaffold of this application.

### BRIEF DESCRIPTION OF THE FIGURES

FIGS. 1A and 1B show a comparison of an aligned polymer nanofiber mesh (FIG. 1B) used in one embodiment of a tissue scaffold of this application with tendon substance (FIG. 1A).

FIGS. 2A and 2B show the experimental design of the growth factor TGF-β3 release (FIG. 2A) and bioactivity studies (FIG. 2B) described in the examples.

FIGS. 3A through 3C show release results from the TGF-β3 release and bioactivity study.

FIGS. 4A through 4C show stability results from the TGF-β3 release and bioactivity study.

FIGS. 5A through 5C show cell growth results from the TGF-β3 release and bioactivity study.

FIGS. 6A through 6C show collagen results from the TGF-β3 release and bioactivity study.

FIGS. 7A through 7C show gene expression results from the TGF-β3 release and bioactivity study.

FIG. 8 is a diagram depicting implantation of a tissue scaffold of this application in integrative rotator cuff repair.

### DETAILED DESCRIPTION

### Definitions

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

Unless defined otherwise, all technical and scientific terms used herein have the meaning commonly understood by a person skilled in the art to which this invention belongs. However, except as otherwise expressly provided herein, each of the following terms, as used in this application, shall have the meaning set forth below.

As used herein, “active agent” shall mean a component incorporated into the fibers of the microfiber or nanofiber mesh which, when released over time, supports alignment, proliferation and matrix deposition of a selected musculoskeletal cell. Examples include, but are in no way limited to growth factors such as transforming growth factor-beta 3(TGF-β3), growth/differentiation factor-5 (gdf-5), bone morphogenetic protein (BMP) 1 through 14, fibroblast growth factor (FGF) and basic fibroblast growth factor (bGF). A single active agent or a combination of active agents may be incorporated into the tissue engineering scaffolds of this application.

As used herein, “aligned fibers” shall mean groups of fibers which are oriented along the same directional axis. Examples of aligned fibers include, but are not limited to, groups of parallel fibers.

As used herein, a “biocompatible” material is a synthetic or natural material used to replace part of a living system or to function in intimate contact with living tissue. Biocompatible materials are intended to interface with biological systems to evaluate, treat, augment or replace any tissue, organ or function of the body. The biocompatible material has the ability to perform with an appropriate host response in a specific application and does not have toxic or injurious effects on biological systems. Nonlimiting examples of biocompatible materials include a biocompatible ceramic, a biocompatible polymer or a biocompatible hydrogel.

As used herein, “biodegradable” means that the material, once implanted into a host, will begin to degrade.

As used herein, “biomimetic” shall mean a resemblance of a synthesized material to a substance that occurs naturally in a human body and which is not substantially rejected by (e.g., does not cause an unacceptable adverse reaction in) the human body. When used in connection with the tissue scaffolds, biomimetic means that the scaffold is substantially biologically inert (i.e., will not cause an unacceptable immune response/rejection) and is designed to resemble a structure (e.g., soft tissue anatomy) that occurs naturally in a mammalian, e.g., human, body and that promotes healing when implanted into the body.

As used herein, “chondrocyte” shall mean a differentiated cell responsible for secretion of extracellular matrix of cartilage.

As used herein, “chondrogenesis” shall mean the formation of cartilage tissue.

As used herein, “effective amount” or “amount effective” shall mean a concentration, combination or ratio of one or more active agents incorporated in the microfiber or nanofiber mesh which when released over time from the substrate supports alignment, proliferation and matrix deposition of a selected musculoskeletal cell. In one embodiment, efficacy of the amount is determined by an increase in collagen I and/or collagen II production, mineralization and/or proteoglycan production by musculoskeletal cells or stem cells seeded on the tissue scaffold.

As used herein, “fibroblast” shall mean a cell which may be mesodermally derived that secretes proteins and molecular collagen including fibrillar procollagen, fibronectin and collagenase, from which an extracellular fibrillar matrix of connective tissue may be formed. Fibroblasts synthesize and maintain the extracellular matrix of many tissues, including but not limited to connective tissue. A “fibroblast-like cell” means a cell that shares certain characteristics with a fibroblast (such as expression of certain proteins).

As used herein, “fibrochondrocyte” shall mean a cell having features of chondrocytes and fibroblasts. Like chondrocytes, they have a rounded morphology and are protected by a territorial matrix. Like fibroblasts, the cells produce collagen-1, and like chondrocytes, these cells can produce collagen-2.

As used herein, “graft” shall mean the device to be implanted during medical grafting, which is a surgical procedure to transplant tissue without a blood supply, including but not limited to soft tissue graft, synthetic grafts, and the like.

As used herein, “matrix” shall mean a three-dimensional structure fabricated from biomaterials. The biomaterials can be biologically-derived or synthetic.

As used herein, “mesh” means a network of material. In one embodiment, the mesh may be woven synthetic fibers, non-woven synthetic fibers, microfibers and nanofibers suitable for implantation into a mammal, e.g., a human. The woven and non-woven fibers may be made according to well known techniques. The microfiber or nanofiber mesh may be made according to techniques known in the art and those disclosed in, e.g., International application no. PCT/US2008/001889 filed on Feb. 12, 2008 to Lu et al., which application is incorporated by reference as if recited in full herein. Fibers of the mesh may be aligned or unaligned.

As used herein, “microfiber” shall mean a fiber with a diameter no more than 1000 micrometers.

As used herein, “nanofiber” shall mean a fiber with a diameter no more than 1000 nanometers.

In one embodiment, the microfibers and/or or nanofibers are comprised of a biodegradable polymer that is electrospun into a fiber. The microfibers and/or nanofibers of the scaffold are oriented in such a way (i.e., aligned or unaligned) so as to mimic the natural architecture of the soft tissue to be repaired. Moreover, the microfibers and/or nanofibers and the subsequently formed microfiber and/or nanofiber scaffold are controlled with respect to their physical properties, such as for example, fiber diameter, pore diameter, and porosity so that the mechanical properties of the microfibers and/or nanofibers and microfiber and/or nanofiber scaffold are similar to the native tissue to be repaired, augmented or replaced. Thus, in the case of a rotator cuff repair, the microfiber and/or nanofiber scaffold is able to regenerate the native insertion of tendon-to-bone through interface tissue engineering and promote tendon-to-bone integration and biological fixation.

As used herein, “musculoskeletal cell” shall mean a chondrocyte, fibrochondrocyte, fibroblast or osteoblast.

As used herein, “osteoblast” shall mean a bone-forming cell which may be derived from mesenchymal osteoprogenitor cells and which forms an osseous matrix in which it becomes enclosed as an osteocyte. The term may also be used broadly to encompass osteoblast-like, and related, cells, such as osteocytes and osteoclasts. An “osteoblast-like cell” means a cell that shares certain characteristics with an osteoblast (such as expression of certain proteins unique to bones), but is not an osteoblast. “Osteoblast-like cells” include preosteoblasts and osteoprogenitor cells.

As used herein, “osteogenesis” shall mean the production of bone tissue.

As used herein, “osteointegrative” means having the ability to chemically bond to bone.

As used herein, “polymer” means a chemical compound or mixture of compounds formed by polymerization and including repeating structural units. Polymers may be constructed in multiple forms and compositions or combinations of compositions.

As used herein, “porosity” means the ratio of the volume of interstices of a material to a volume of a mass of the material. As used herein, “porous” shall mean having an interconnected pore network.

The “rotator cuff” refers to the group of muscles and tendons that surround the humeral head. Specifically, the rotator cuff consists of a group of four muscles and tendons, including the supraspinatus, infraspinatus, teres minor, and subscapularis, which function in synchrony to stabilize the glenohumeral joint as well as to actively control shoulder kinematics. The supraspinatus tendon inserts into the humeral head via a direct insertion exhibiting region-dependent matrix heterogeneity and mineral content.

As used herein, “soft tissue graft” shall mean a graft which is not synthetic, and can include autologous grafts, syngeneic grafts, allogeneic grafts, and xenogeneic graft. As used herein, “soft tissue” includes, as the context may dictate, tendon and ligament, as well as the bone to which such structures may be attached. Preferably, “soft tissue” refers to tendon- or ligament-bone insertion sites requiring surgical repair, such as for example tendon-to-bone fixation.

As used herein, “stem cell” means any unspecialized cell that has the potential to develop into many different cell types in the body, such as mesenchymal osteoprogenitor cells, osteoblasts, osteocytes, osteoclasts, chondrocytes, chondrocyte progenitor cells, fibrochondrocytes, fibroblasts and fibroblast progenitor cells. Nonlimiting examples of “stem cells” include mesenchymal stem cells, embryonic stem cells and induced pluripotent cells.

As used herein, “synthetic” shall mean that the material is not of a human or animal origin.

As used herein, all numerical ranges provided are intended to expressly include at least the endpoints and all numbers that fall between the endpoints of ranges.

The following embodiments are provided to further illustrate the tissue scaffold production of this application. These embodiments are illustrative only and are not intended to limit the scope of this application in any way.

### Embodiments

Four distinct yet continuous tissue regions are observed at the tendon-bone junction: tendon proper, non-mineralized fibrocartilage, mineralized fibrocartilage and bone (Benjamin et al. J. Anat. 1986 149:89-100; Benjamin et al. Comp Biochem. Physiol A. MoI. Integr. Physiol. 2002 133(4):931-945; Woo et al. (1988) “Ligament, Tendon, and Joint Capsule Insertions to Bone. In Injury and Repair of the Musculoskeletal Soft Tissues.” Woo, S L, Bulkwater, J A, eds., American Academy of Orthopaedic Surgeons: Savannah, Ga., pp. 133-166). The tendon proper consists of fibroblasts found between aligned collagen fibers in a matrix rich in collagen I, with small amounts of collagen III and proteoglycans (Blevins et al. Orthop. Clin. North Am. 1997 28(1):1-16). The non-mineralized and mineralized fibrocartilage consists of aligned collagen fibers: the non-mineralized fibrocartilage region is composed of fibrochondrocytes in a matrix of collagen I, II, and III with fibers oriented perpendicular to the calcified interface region (Kumagai et al. J. Anat. 1994 185(Pt. 2):279-284); the mineralized fibrocartilage region consists of hypertrophic fibrochondrocytes within a matrix of collagen I and II (Kumagai et al. J. Anat. 1994 185(Pt. 2):279-284) as well as collagen X (Thomopoulos et al. J. Orthop. Res. 2003 21:413). The last region of the insertion site is bone which consists of osteoblasts, osteoclasts, and osteocytes in a mineralized matrix rich in type I collagen. This controlled matrix heterogeneity exhibited by the tendon-bone interface serves to minimize stress concentrations and to mediate load transfer between two distinct tissue types (Thomopoulos et al. J. Orthop. Res. 2003 21:413; Woo et al. (1988) “Ligament, Tendon, and Joint Capsule Insertions to Bone. In Injury and Repair of the Musculoskeletal Soft Tissues.” Woo, S L, Bulkwater, J A, eds., American Academy of Orthopaedic Surgeons: Savannah, Ga., pp. 133-166). Due to its functional significance, interface regeneration is a pre-requisite for biological fixation.

Musculoskeletal injuries such as rotator cuff tears often occur at the tendon-bone interface. Current repair methods result in scar tissue formation and poor tendon-bone integration (Galatz L. J Orthop Res 2007 25:1621-1628; Benjamin, M and Ralphs, J. R. J. Anat. 1998 193(Pt 4):481-494). The native insertion site is composed mainly of collagen types I, II, X, and proteoglycans, and regeneration of the interface is a prerequisite for the biological fixation of tendon grafts. Growth factors play an important role in this process (Kovacevic D. and Rodeo, S. A. Clin Orthop Relat Res 2008 466(3):622-633; Rodeo S A. J Shoulder Elbow Surg 2007 16(5S):191S-7S). For example, TGF-β3 has been reported to be upregulated during the formation of the tendon-bone insertion (Galatz L. J Orthop Res 2007 25:1621-1628).

Described in this application are biomimetic tissue scaffolds for treatment of musculoskeletal injuries designed for tendon-bone integration. These tissue scaffolds promote interface regeneration upon controlled release of one or more active agents which support alignment, proliferation and/or matrix deposition of a selected musculoskeletal cell.

In one embodiment, the tissue scaffold comprises a first phase of polymer microfiber or nanofiber mesh and an active agent released over time from the first phase which supports alignment, proliferation and matrix deposition of a selected musculoskeletal cell. The microfibers and/or nanofibers of the first phase are oriented in such a way (i.e., aligned or unaligned) so as to mimic the natural architecture of the soft tissue to be repaired.

In another embodiment, the tissue scaffold is biphasic or multiphasic, thus comprising one or more additional phases of polymer microfiber or nanofiber mesh and an active agent released over time from the one or more additional phases which supports alignment, proliferation and matrix deposition of a selected musculoskeletal cell. One aspect of such biphasic or multiphasic microfiber or nanofiber scaffolds is that each phase is “continuous” with the phase adjacent to it. Thus, in the tissue scaffolds of this disclosure, the interface between one phase and the next is designed, e.g., by electrospinning, conventional extrusion and/or 3-D printing techniques, to mimic the natural anatomical transition between, e.g., tendon and bone at a tendon-to-bone interface. By designing the tissue scaffolds of the present disclosure so that the phases are continuous, improved fixation and function is achieved by minimizing stress concentrations and mediating load transfer between tendon and bone compared to prior systems.

The microfibers and/or nanofibers of the one or more additional phases are oriented in such a way (i.e., aligned or unaligned) so as to mimic the natural architecture of the soft tissue to be repaired.

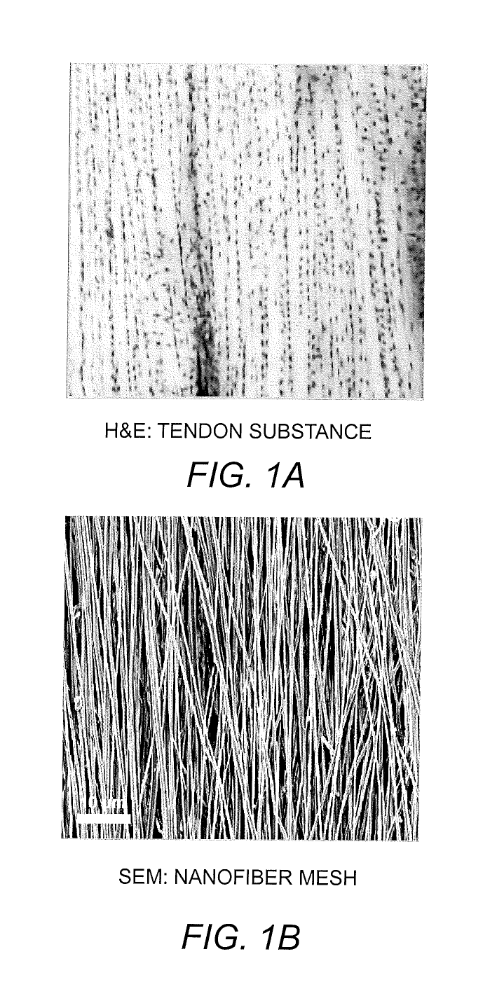

The polymer microfiber and/or nanofiber mesh used in these tissue scaffolds is advantageous for soft tissue repair and tissue engineering. In particular, the fiber diameters mimic collagen fibrils and the matrix organization resembles tendon ECM (see FIGS. 1A and 1B). Further the polymer microfiber and/or nanofiber mesh provides for high porosity and high surface area-to-volume ratio. In addition, the mechanical properties are easily controlled.

The tissue scaffold further comprises one or more active agents incorporated into the tissue scaffold which support alignment, proliferation and/or matrix deposition of a selected musculoskeletal cell. In one embodiment, the active agent or agents is incorporated in an amount effective to increase collagen I production, collagen II production, mineralization and/or proteoglycan production of the selected musculoskeletal cell. Examples of active agents useful in these tissue scaffolds include, but are in no way limited to, growth factors such as transforming growth factor-beta 3(TGF-β3), growth/differentiation factor-5 (gdf-5), bone morphogenetic protein (BMP) 1 through 14, fibroblast growth factor (FGF) and basic fibroblast growth factor (bGF). Tissue scaffolds of this application may include a single active agent or a combination of active agents. The amount of active agent incorporated into the scaffold will vary depending upon the agent selected, the musculoskeletal cells seeded on the scaffold and or the injury to be treat. In general, however, the active agent will be in the range of 0.0001-10% based upon polymer weight depending upon the active agent selected.

For multiphasic tissue scaffolds of this application, the first phase and the one or more additional phases may release the same active agent or agents at the same concentration, they may release the same active agent or agents at different concentrations, or they may release different active agents.

The active agent or active agents selected to be incorporated into the first phase or additional one or more phases of the tissue scaffold is based upon the musculoskeletal cell seeded on that phase of the tissue scaffold. For chondrocytes, for example, the active agent selected for incorporation may be TGF-β3 as this growth factor promotes chondrocyte proliferation, chondrogenic matrix synthesis and relevant gene expression (Na et al. J. Biotechnol 2007 128:412-22; Lima et al. Osteoarthritis Cartilage 2007 15: 1025-1033 and Choi et al. J. Biomed. Mater. Res. Part A 2007 83A(4):897-905). For osteoblastic differentiation of stem cells, for example, the active agent selected for incorporation may be BMP-2. In this embodiment, dexamethasone may also be added. For fibroblastic differentiation of stem cells, the active agent selected for incorporation may be bFGF.

In one embodiment, one or more phases of the tissue scaffold may further comprise a purified albumin such as, but not limited to, bovine serum albumin (BSA). A purified albumin can be incorporated into one or more phases of the tissue scaffold in an amount ranging from 0 to 20% based upon polymer weight. Without being limited to any particular theory, it is believed that albumin inhibits adsorption of active agents such as TGF-β3 to the polymer. Further, addition of albumin stabilizes the active. For example, BSA is effective in preserving the α-helical structure of TGF-β3.

The tissue scaffolds of this application can be engineered to remain in place for as long as the treating physician deems necessary. Typically, the microfiber and/or nanofiber scaffold is engineered to biodegrade between 6-18 months after implantation, such as for example 12 months. Examples of polymers which can be selected for the polymer microfiber and/or nanofiber mesh include, but are not limited to, biodegradable polymers selected from the group consisting of aliphatic polyesters, poly(amino acids), modified proteins, polydepsipeptides, copoly(ether-esters), polyurethanes, polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, modified polysaccharides, polycarbonates, polytyrosinecarbonates, polyorthocarbonates, poly(trimethylene carbonate), poly(phosphoester)s, polyglycolide, polylactides, polyhydroxybutyrates, polyhydroxyvalerates, polydioxanones, polyalkylene oxalates, polyalkylene succinates, poly(malic acid), poly(maleic anhydride), polyvinylalcohol, polyesteramides, polycyanoacrylates, polyfumarates, poly(ethylene glycol), polyoxaesters containing amine groups, poly(lactide-co-glycolides), poly(lactic acid)s, poly(glycolic acid)s, poly(dioxanone)s, poly(alkylene alkylate)s, biopolymers, collagen, silk, chitosan, alginate, and a blend of two or more of the preceding polymers. In one embodiment, the polymer comprises at least one of poly(lactide-co-glycolide) or poly-caprolactone. In one embodiment, the polymer is a copolymer, such as for example a poly(D,L-lactide-co-glycolide (PLGA) and/or poly-caprolactone (PCL).

Selection of a polymer or polymers used in the microfiber and/or nanofiber mesh is based upon the length of time the scaffold is needed to remain in place as well as the polymer's degradation characteristics which control release of the active agent or agents from the scaffold. For example, a polymer such as PLGA is bulk-eroding while a polymer such as PCL is surface eroding. By using only a bulk-eroding polymer or only a surface eroding polymer or combining both of these types of polymers into a polymer microfiber and/or nanofiber mesh, release of the active agent or agents from the tissue scaffold can be controlled and a temporal gradient of release of the active agent or agents supportive of alignment, proliferation and/or matrix deposition of a selected musculoskeletal cell can be created.

A spatial gradient of release of the active agent or agents can also be generated by including varying concentrations of the active agent in the polymers. For example, the first phase may contain an active agent such as a growth factor at a concentration of 1% while an additional phase may contain the growth factor at a concentration of 2%.

In one embodiment, the active agent is incorporated into the polymer microfiber or nanofiber mesh by electrospinning of the polymer microfibers or nanofibers. In this embodiment, a spatial gradient of the active agent or agents can be generated by layering the polymer in different phases during electrospinning (i.e. first phase—active agent concentration of 1%, additional phase or phases—active agent concentration of 2%, 3% and so forth).

In some embodiments, additional components may be added to one or more phases of the tissue scaffold to further support alignment, proliferation and matrix deposition of a selected musculoskeletal cell seeded on that phase of the tissue scaffold. Examples of additional components include, but are in no way limited to calcium phosphate, glass and/or glass ceramics. In one embodiment, hydroxyapatite “HA” nano-particles may be added to PLGA to form a composite and a phase of the tissue scaffold which mimics the calcified fibrocartilage interface.

The tissue scaffold is seeded with a selected musculoskeletal cell or stem cells which differentiate into the selected musculoskeletal cell. Examples of musculoskeletal cells which can be seeded onto these scaffolds include chondrocytes, fibrochondrocytes, fibroblasts and osteoblasts. Tissue scaffolds of this application may be seeded with a single type of musculoskeletal cell, a mixture of selected musculoskeletal cells and/or stem cells which differentiate into a selected musculoskeletal cell or mixture of selected musculoskeletal cells. For multiphasic tissue scaffolds, the first phase and the one or more additional phases may be seeded with the same selected musculoskeletal cell or mixture of cells, and/or stem cells which differentiate into the selected musculoskeletal cell or mixture of cells. Alternatively, the first phase and the one or more additional phases may seeded with different selected musculoskeletal cells.

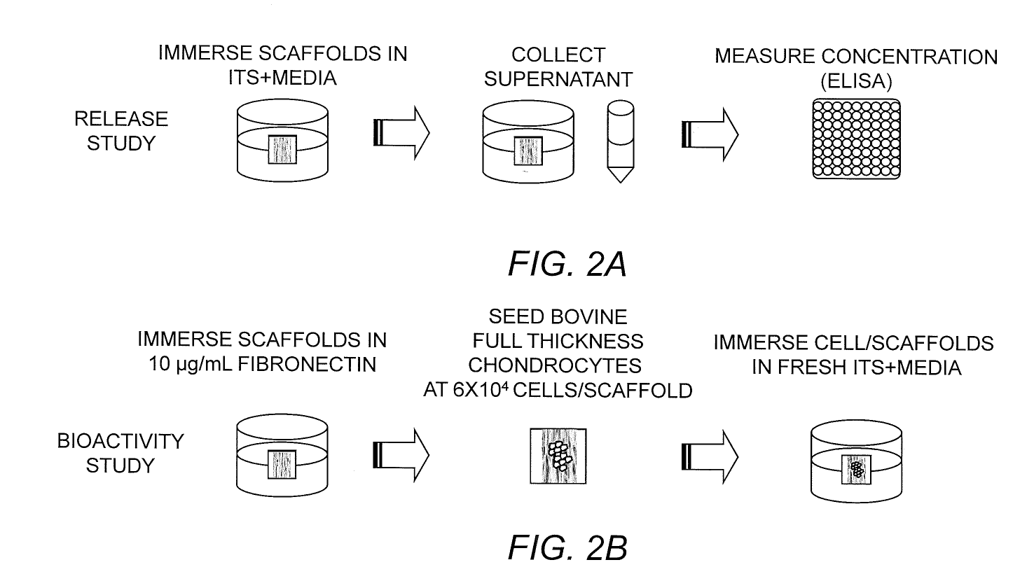

A tissue scaffold in accordance with this application comprising aligned polylactide-co-glycolide (PLGA) nanofiber mesh and the active agent TGF-β3 was prepared and the effect of its controlled release on chondrocyte response was evaluated. The experimental design for this evaluation is shown in FIGS. 2A and 2B. Scaffolds of this application including aligned polylactide-co-glycolide (PLGA) nanofiber mesh with the active agent TGF-β3 and aligned polylactide-co-glycolide (PLGA) nanofiber mesh with the active agent TGF-β3 and BSA were compared to a PLGA nanofiber mesh scaffold with exogenous addition of TGF-β3 (PLGA-EXO).

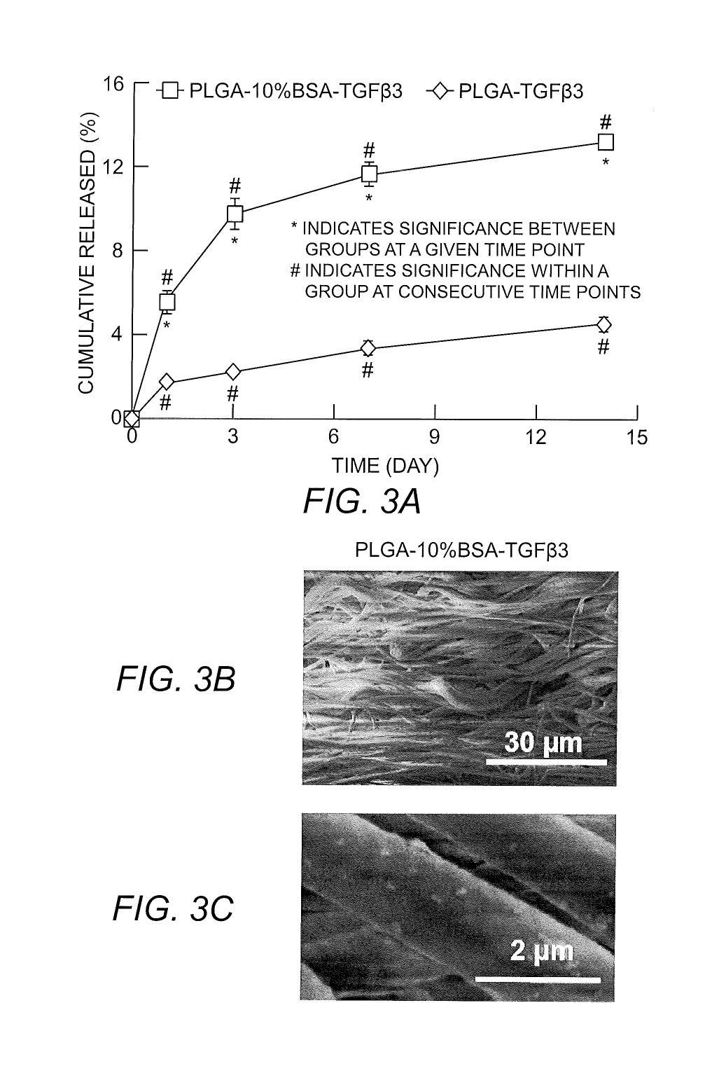

As shown in FIGS. 3A through 3C, growth factor release was consistent over time. In addition, release kinetics was modulated by BSA (FIG. 3A).

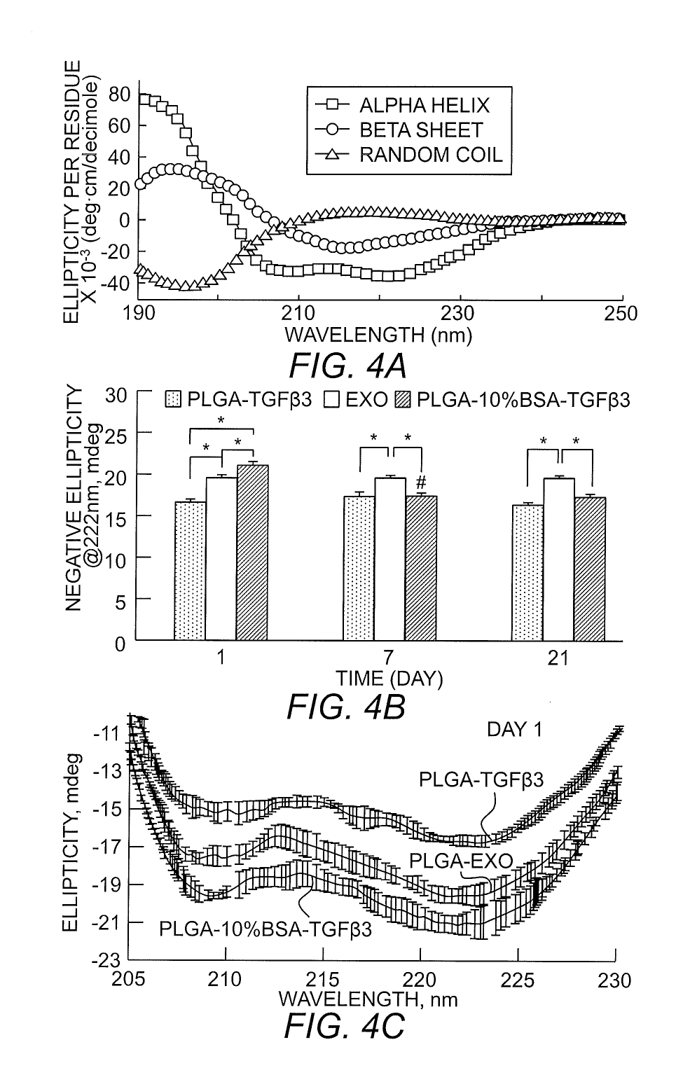

Ellipticity (degree of folding of the protein structure) was detected using Circular Dichroism Spectroscopy. The data showed preservation of growth factor structure indicative of its stability. See FIGS. 4A through 4C.

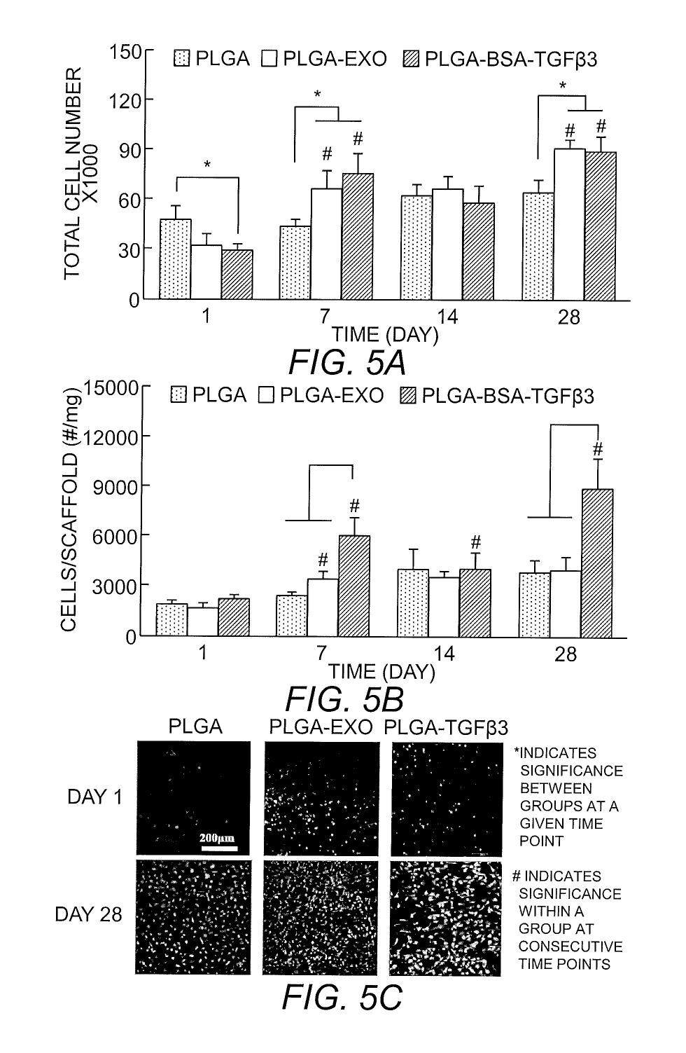

Further, significantly higher cell growth was observed on PLGA-EXO and PLGA-BSA-TGF-β3 scaffolds on Day 7 and Day 28 (see FIGS. 5A through 5C).

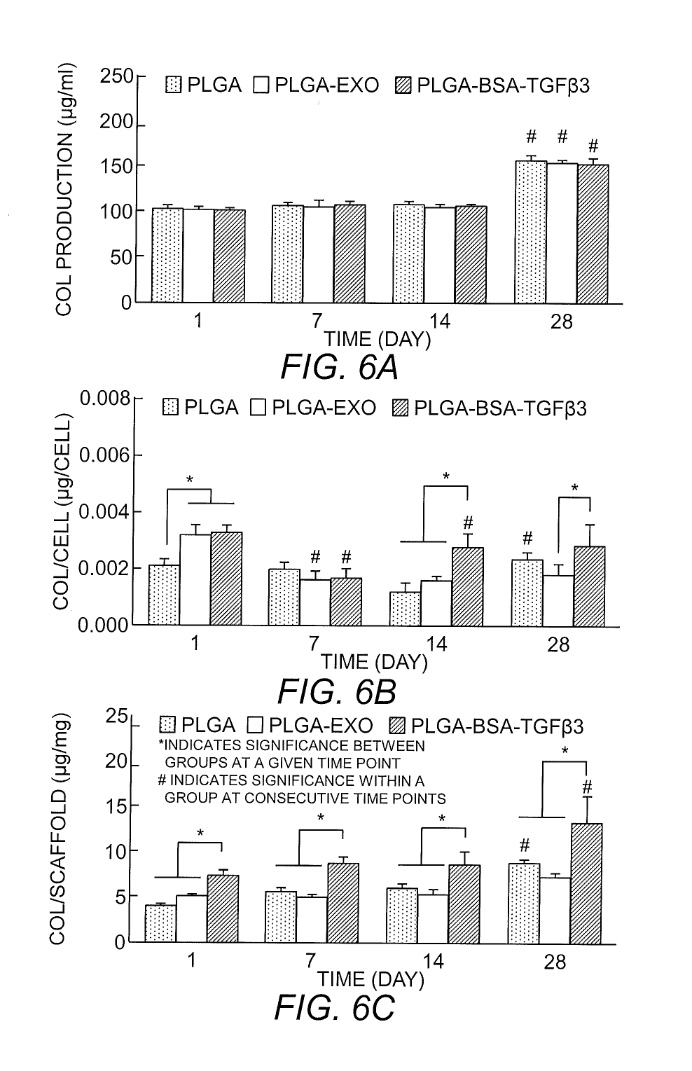

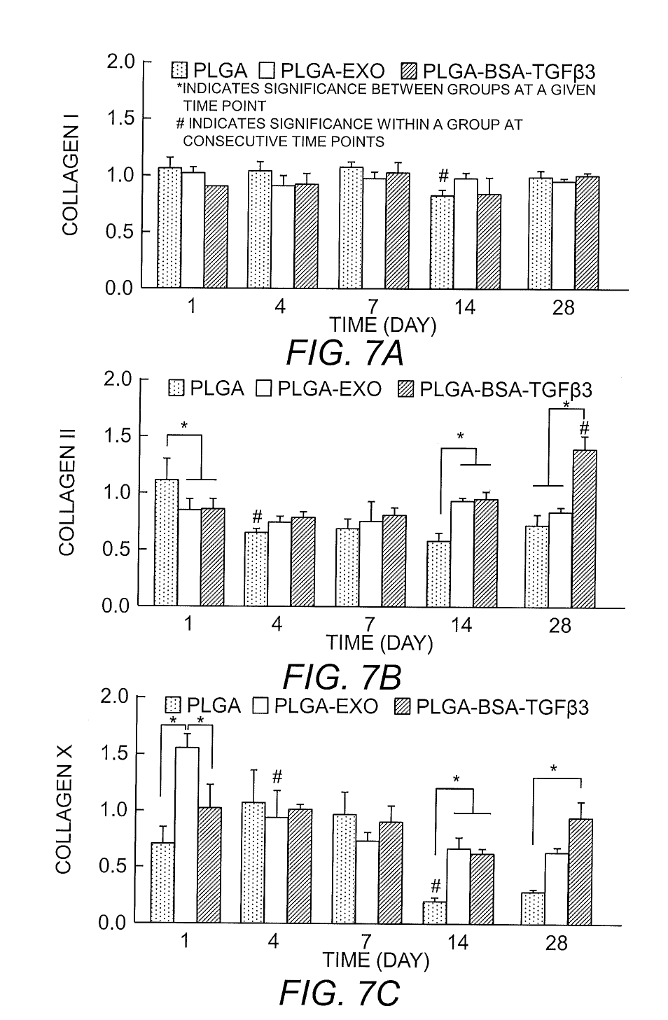

Total collagen was found to increase over time for all scaffolds tested. However, total collagen was significantly higher for PLGA-TGFβ3 over PLGA alone and PLGA-EXO. Accordingly, the PLGA-BSA-TGF-β3 scaffold of the instant application actually performed better than the exogenous positive control (see FIGS. 6A through 6C). Gene expression analysis showed that while collagen I expression was maintained for all groups, significantly higher collagen II and collagen X expressions were found for the PLGA-BSA-TGF-β3 group (FIGS. 7A through 7C).

Thus, as shown be this evaluation, TGF-β3 released from a tissue scaffold of the instant application was bioactive and promoted chondrocyte proliferation and biosynthesis. The upregulation of expression of Col II and X genes in the presence of eluted TGF-β3 were also documented effects of this growth factor on chondrocytes. Further, the controlled release of TGF-β3 achieved through use of a tissue scaffold of the instant application was significantly more effective than exogenous TGF-β3 control in promoting both cell growth and matrix production.

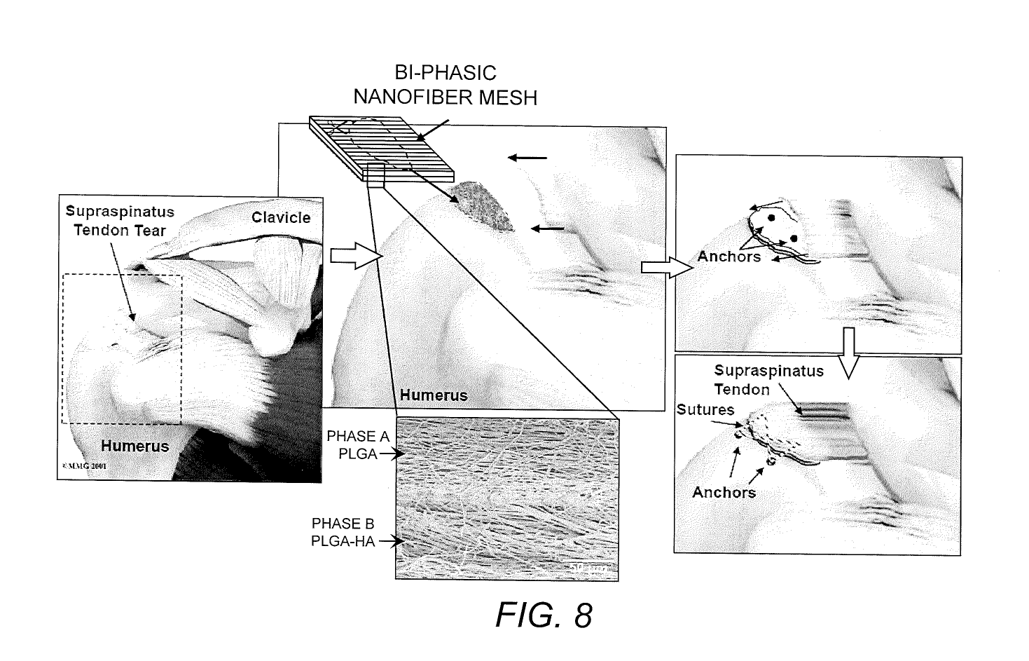

Accordingly, the tissue scaffolds of this application are expected to be useful in integrative tendon-bone repair and thus provide a treatment for various musculoskeletal injuries. In one embodiment, a biomimetic scaffold designed according to the various embodiments described herein can be used to enhance biological fixation and mechanical stability at a rotator cuff repair site. One particular embodiment of such a scaffold in the form of a “graft patch” is depicted in FIG. 8.

Throughout this application, certain publications are referenced. The disclosures of these publications are hereby incorporated by reference into this application in order to more fully describe the state of the art as of the date of the invention described and claimed herein.

The following section provides further illustration of the methods and apparatuses of the present invention. These examples are illustrative only and are not intended to limit the scope of the invention in any way.

### EXAMPLES

### Example 1

### Scaffold Fabrication

Nanofiber scaffolds of PLGA were fabricated by electrospinning a solution of PLGA in N,N-dimethylformamide (DMF, Sigma-Aldrich, St. Louis, Mo.). Briefly, PLGA was mixed with DMF and ethyl alcohol. The polymer solution was loaded into a 5-mL syringe with a 18.5-gauge stainless steel blunt-tip needle and electrospun at 8 to 10 kV on a rotating mandrel (20 m/s) for aligned scaffolds. For unaligned scaffolds, the mandrel was stationary. The polymer solution was dispensed using a syringe pump. The PLGA/TGF-β3 scaffolds were fabricated by adding TGF-β3 into the PLGA solution prepared as described above and electrospinning it under the same conditions. The PLGA/BSA and PLGA/BSA/TGF-β3 nanofiber scaffolds were produced by electrospinning of PLGA solution prepared as described above that is also containing pulverized bovine serum albumin and TGF-β3, respectively, under the same conditions.

### Example 2

### TGF-β3 Release

Release was assessed in serum-free ITS+(Universal Culture Supplement Premix (BD Biosciences, San Jose, Calif.) media, and TGF-β3 concentration was measured with ELISA (n=6, R&D Systems). Stability (n=3) of TGF-β3 released was evaluated using circular dichroism (CD) analysis.

### Example 3

### Cell Culture

Chondrocytes enzymatically digested from bovine calves (6×10⁴ cells/cm²) were cultured on scaffolds in serum-free ITS+ media. Experimental group included PLGA-TGFβ3 scaffolds, while cells cultured on PLGA and on PLGA with exogenous TGF-β3 (1 ng/mL) served as controls. Viability was evaluated with confocal microscopy (n=3).

### Example 4

### End-Point Analyses (1, 7, 14, 28 Days)

Total DNA (n=6) was quantified by Picogreen Assay. Glycosaminoglycan (GAG) and collagen syntheses (n=6) were assessed by dimethylmethylene blue (DMMB) and Sircol assays, respectively. Gene expressions (Col I, II, and X) were analyzed by RT-PCR (n=4).

### Example 5

### Statistical Analysis

Results are given as Mean±STD. ANOVA and Tukey-Kramar post-hoc test was used for all pair-wise comparisons.

1. A tissue scaffold for treating a musculoskeletal tissue injury, said tissue scaffold comprising a first phase of polymer microfiber or nanofiber mesh and an active agent released over time from said first phase which supports alignment, proliferation and matrix deposition of a selected musculoskeletal cell.

2. The tissue scaffold of claim 1 wherein said active agent is incorporated into the tissue scaffold in an amount effective to increase collagen I production, collagen II production, mineralization and/or proteoglycan production of the selected musculoskeletal cell.

3. The tissue scaffold of claim 1 wherein said active agent is selected from the group consisting of TGF-β3, gdf-5, BMP1, BMP2, BMP3, BMP4, BMP5, BMP6, BMP7, BMP8, BMP9, BMP10, BMP11, BMP12, BMP13, BMP14, FGF and bFGF or any combination thereof.

4. The tissue scaffold of claim 1 further comprising an albumin.

5. The tissue scaffold of claim 1 seeded with a selected musculoskeletal cell or stem cells which differentiate into the selected musculoskeletal cell.

6. The tissue scaffold of claim 1 further comprising one or more additional phases of polymer microfiber or nanofiber mesh and an active agent released over time from said one or more additional phases which supports alignment, proliferation and matrix deposition of a selected musculoskeletal cell.

7. The tissue scaffold of claim 6 wherein said active agent is selected from the group consisting of TGF-β3, gdf-5, BMP1, BMP2, BMP3, BMP4, BMP5, BMP6, BMP7, BMP8, BMP9, BMP10, BMP11, BMP12, BMP13, BMP14, FGF and bFGF or any combination thereof.

8. The tissue scaffold of claim 6 wherein said first phase and said one or more additional phases release the same active agent.

9. The tissue scaffold of claim 6 wherein said first phase and said one or more additional phases comprise the same active agent at different concentrations.

10. The tissue scaffold of claim 6 wherein said first phase and said one or more additional phases release the different active agents.

11. The tissue scaffold of claim 6 wherein said first phase and said one or more additional phases are seeded with the same selected musculoskeletal cell or stem cells which differentiate into the selected musculoskeletal cell.

12. The tissue scaffold of claim 6 wherein said first phase and said one or more additional phases are seeded with different selected musculoskeletal cells.

13. The tissue scaffold of claim 6 wherein said one or more additional phases further comprises calcium phosphate, glass and/or a glass ceramic.

14. The tissue scaffold of claim 6 wherein said one or more additional phases further comprises hydroxyapatite.

15. The tissue scaffold of claim 1 wherein said polymer microfiber or nanofiber mesh comprises a bulk eroding polymer, a surface eroding polymer or a combination thereof.

16. The tissue scaffold of claim 1 wherein said active agent is incorporated into said polymer microfiber or nanofiber mesh by electrospinning.

17. A method for producing a tissue scaffold which promotes musculoskeletal cell proliferation, alignment and/or matrix production, said method comprising seeding a selected musculoskeletal cell onto a tissue scaffold comprising a first phase of polymer microfiber or nanofiber mesh and an active agent released over time from the first phase which supports alignment, proliferation and matrix deposition of the selected musculoskeletal cells.

18. The method of claim 17 wherein said tissue scaffold comprises one or more additional phases of polymer microfiber or nanofiber mesh and an active agent released over time from said one or more additional phases which supports alignment, proliferation and matrix deposition of a selected musculoskeletal cell.

19. The method of claim 18 wherein said selected musculoskeletal cells seeded onto said one or more additional phases of the tissue scaffold are different from said selected musculoskeletal cells seeded onto said first phase.

20. The method of claim 18 wherein said phases of said tissue scaffold comprise different active agents.

21. The method of claim 17 wherein said polymer microfiber or nanofiber mesh with said active agent incorporated therein is produced by electrospinning.

22. The method of claim 17 wherein said active agent is incorporated into said tissue scaffold in an amount effective to increase collagen I production, collagen II production, mineralization and/or proteoglycan production of said selected musculoskeletal cell.

23. The method of claim 22 wherein said active agent is selected from the group consisting of TGF-β3, gdf-5, BMP1, BMP2, BMP3, BMP4, BMP5, BMP6, BMP7, BMP8, BMP9, BMP10, BMP11, BMP12, BMP13, BMP14, FGF and bFGF or any combination thereof.

24. The method of claim 17 wherein an albumin is added to said tissue scaffold.

25. A tissue scaffold for treatment of a musculoskeletal tissue injury produced in accordance with the method of claim 17.

26. A method for treating a musculoskeletal tissue injury in a subject comprising surgically implanting in the subject at a site of musculoskeletal injury a tissue scaffold of claim 1.
